(19) United States
(12) Patent Application Publication
(10) Pub. No.: US 2026/0014068 A1
(43) Pub. Date: Jan. 15, 2026

(54) HYDROGEL BASED ON ZINC GLUCONATE AND HYALURONIC ACID ESTERS

(71) Applicant: BMG PHARMA S.P.A., Milano (IT)

(72) Inventors: Marco Mastrodonato, Milano (IT); Luca Stucchi, Torviscosa (UD) (IT); Alessandra Sechi, Torviscosa (UD) (IT); Fabrizio Picotti, Torviscosa (UD) (IT); Rita Gianni, Monrupino (TS) (IT)

(73) Assignee: BMG PHARMA S.P.A., Milano (IT)

(21) Appl. No.: 19/335,006

(22) Filed: Sep. 22, 2025

Related U.S. Application Data
(62) Division of application No. 17/754,150, filed on Mar. 25, 2022.

(30) Foreign Application Priority Data
Sep. 27, 2019 (IT) 102019000017387

(51) Int. Cl. A61K 8/73; A61K 8/04; A61K 8/27; A61K 31/728; A61K 33/30; A61K 45/06; A61L 27/20; A61L 27/52; A61Q 19/08

(52) U.S. Cl. CPC A61K 8/735; A61K 8/042; A61K 8/27; A61K 31/728; A61K 33/30; A61K 45/06; A61L 27/20; A61L 27/52; A61Q 19/08; A61K 2800/91

(57) ABSTRACT

Disclosed is a hydrogel containing zinc gluconate and hyaluronic acid esters, a process for the preparation thereof, compositions containing it, and the use of the hydrogel and the compositions thereof in the pharmaceutical and cosmetic fields or as topical or injectable medical devices.

This utility application is a divisional of U.S. Ser. No. 17/754,150, filed Mar. 25, 2022, which is a U.S. National Stage of PCT/EP2020/076840, filed 25 Sep. 2020, which claims priority to and the benefit of Italian Patent Application No. 102019000017387 filed 27 Sep. 2019, the contents of which are all incorporated herein by reference in their entireties.

The present invention relates to a hydrogel containing zinc gluconate and hyaluronic acid esters, a process for the preparation thereof, compositions containing it, and the use of the hydrogel and the compositions thereof in the pharmaceutical and cosmetic fields or as topical or injectable medical devices.

### STATE OF THE ART

Zinc is a trace element which is essential to the body. It is involved in proinflammatory cytokine modulation mechanisms, and exhibits radical scavenger activity against reactive oxygen species (ROS) [A. S. Prasad, Frontiers in Nutrition, 2014, Vol. 1 pp. 1-10]. It plays an important part in disorders such as osteoarthritis involving massive production of free radicals, and low zinc levels have been found in patients suffering from said disorder [A. Mierzecki, Biol Trace Elem. Res., 2011, 143, pp. 854-862]. Zinc gluconate [WO 2016/141946] is used for its antibacterial properties and its effect on accelerating the wound-healing process. Zinc is usually administered orally, absorbed in the intestine, and rapidly sequestered in the plasma by proteins; it has a very fast turnover, and does not accumulate in the body [M. Jarosz, Inflammopharmacol, 2017, 25, pp. 11-24].

Hyaluronic acid is a glycosaminoglycan consisting of repeating units of glucuronic acid and N-acetylglucosamine bonded together, alternatively, via glycoside bonds β1→4 and β1→3. It is an essential element of connective tissue, and is also present in synovial fluid, vitreous humour and the umbilical cord.

WO2009/098127 discloses a biocompatible injectable product for zinc delivery containing, dispersed in a matrix, a saccharide zinc salt such as zinc hyaluronate or zinc gluconate. The product can be used in medical devices or in medicinal or cosmetic preparations, e.g. for the treatment of wrinkles, arthritis and inflammation.

Hyaluronic acid lipoate ester or lipoate/formate ester is an ester derivative wherein the hydroxyl groups of hyaluronic acid are esterified with residues of lipoic acid or lipoic and formic acid, with different degrees of substitution (DS). DS means the number of hydroxyls involved in an ester bond with lipoate or lipoate/formate residues per repeating disaccharide unit of hyaluronic acid. Hyaluronic acid lipoate/formate ester is known to have anti-inflammatory, antioxidant and skin-protecting properties (WO2009080220), and its use in the trichology field has also been reported (WO2012080223).

Lipoic acid (or thioctic acid) is a natural molecule, isolated in mammal livers, which acts as an essential cofactor for many enzymatic reactions, including the conversion of pyruvate to acetyl-CoA in the Krebs cycle. The lipoic acid in the body governs the production of antioxidant vitamins C and E, and glutathione. It also exhibits radical scavenger activity in the lipid tissues. It has a high affinity for metals, with which it forms stable, water-insoluble complexes [J. Fuchs “Lipoic acid in health and disease”]. The affinity of lipoic acid for transition metals, in particular zinc, allows the preparation of a system that prolongs the residence time of the metal in the body, and the performance of its favourable biological activities in terms of anti-inflammatory activity and free-radical absorption. However, the low water-solubility of this complex makes said use very difficult.

The purpose of the present invention is to provide a system particularly suitable for delivering zinc and characterised by optimum viscoelastic properties for topical and injectable (mesodermal, subdermal and intra-articular) applications which are useful in the cosmetic and pharmaceutical fields or in medical devices.

### DESCRIPTION OF THE INVENTION

The object of the present invention is a hydrogel containing:
hyaluronic acid esterified on the free hydroxyl groups with lipoic acid or lipoic acid and formic acid, or a pharmaceutically acceptable salt thereof;
zinc gluconate.

The use of a strongly hydrophilic carrier such as hyaluronic acid or a salt thereof, modified by introducing lipoate residues, and optionally formate residues, with an esterification reaction at the level of the hydroxyl functions, constitutes the ideal system for the formation of a complex between the lipoic residue and zinc which is soluble in an aqueous solvent. The absence of a carboxyl residue of lipoic acid, normally involved in the formation of the complex with the metal, as it is engaged in esterification at the level of the hyaluronic acid hydroxyls, is obviated by using the salt between zinc and gluconic acid. As the hyaluronic acid carboxyl group is not involved, the water-solubility remains unchanged.

The esterified hyaluronic acid according to the invention preferably has a molecular weight ranging between 1 kDa and 4×10³ kDa.

According to a preferred embodiment, the number of lipoic acid residues per GlcNAc-GlcUA disaccharide unit of hyaluronic acid ranges between 0.01 and 0.5, while the number of formic acid residues, again per GlcNAc-GlcUA disaccharide unit of hyaluronic acid, ranges between 0 and 0.1.

According to a further preferred embodiment, the amount of zinc gluconate ranges between 0.1 and 25 by weight compared with sodium hyaluronate lipoate or sodium hyaluronate lipoate/formate.

The esterified hyaluronic acid preferably takes the form of a pharmacologically acceptable salt, more preferably the sodium salt.

The characteristics and preparation of hyaluronic acid lipoic and formic esters are described in WO2009/080220, which is incorporated in full herein for reference. As regards synthesis, particular reference is made to pages 5, line 20 to 7, line 5 and examples 1-5 of WO2009/080220.

Another aspect of the present invention relates to a process for the preparation of the hydrogel comprising mixing in water of esterified hyaluronic acid or a salt thereof and zinc gluconate, until a viscous solution is obtained which is subsequently left to stand for a time ranging from 1 to 48 hours, during which the viscoelastic solution typical of a hydrogel is formed. The process can also include a hydrogel sterilisation step. In a preferred embodiment, the products are mixed at a temperature ranging between 20° C. and 30° C.

Another aspect of the invention relates to a hydrogel obtained by the process described above.

Further aspects of the invention relate to a pharmaceutical or cosmetic composition, supplement or medical device containing the hydrogel described herein, optionally combined with compounds or substances having biological activity such as anaesthetics, and in particular lidocaine.

The composition or device has a form or configuration suitable for topical, ophthalmic or injectable administration or application of the hydrogel, in particular intradermal, mesodermal or intra-articular administration. For topical applications the hydrogel is preferably formulated as an oil-in-water (O/W) or water-in-oil (W/O) emulsion, or a gel, foam or unguent.

The uses of the hydrogel are correlated with the presence of various biologically active ingredients; the restorative and maintenance activities typical of sodium hyaluronate are combined with the antioxidant properties of lipoic acid and the various biological activities of zinc gluconate, in terms of (i) interactions with proteins, in particular thioneins (Antioxidant-like properties of Zinc In Activated Endothelial Cells. Hennig B and McClain G J, Journal of the American College of Nutrition, 18(2):152-158. 1999) and enzymes, such as 5-α-reductase (Effect of a topical erythromycin-zinc formulation on sebum delivery. Evaluation by combined photometric-multi-step samplings with Sebutape. Pierard G E and Pierard-Franchimont C, Clinical and Experimental Dermatology, 18(5):410-413. 1993); (ii) anti-irritant activity and reduction of oxidative stress (Antioxidant-like properties of Zinc In Activated Endothelial Cells. Hennig B and McClain G J, Journal of the American College of Nutrition, 18(2):152-158. 1999).

In particular, the compositions usable as topical medical devices can be employed to treat skin disorders such as acne, by reducing sebum production due to the modulating activity of zinc gluconate on 5-α-reductase; rashes and burns, due to the combined presence of anti-irritant agents such as sodium hyaluronate lipoate ester and zinc gluconate.

The hydrogel and the compositions to which the patent relates can have ophthalmic applications due to the viscoelastic and wetting characteristics of hyaluronic acid and the antioxidant characteristics of lipoic acid.

The rheological characteristics of the hydrogel, in particular the reversibility of its viscoelastic structure after imposition of a force, combined with the antioxidant, lubricant and regenerating properties of its ingredients, make the hydrogel according to the invention extremely useful in injectable medical devices as a dermal filler, or for viscosupplementation in the joints. The hydrogel and the corresponding compositions can therefore be advantageously administered by the injectable route into the joint capsule, or to the dermis, wherein they perform a protective action. In addition, the special molecular structure of the polysaccharide, containing lipoate residues bonded via an ester bond to the polymer chain, combined with the interaction of said residues with zinc gluconate which creates the viscoelastic system, modifies the three-dimensional structure of the polymer, making it more resistant to enzymatic attack by hyaluronidase than a crosslink consisting of mere covalent interactions typical of the systems currently available on the market.

Moreover, the hydrogel and the corresponding compositions can be used in the cosmetic field due to the combination of the emollient and moisturising characteristics of the polymer ingredient and the soothing and regenerating characteristics of the combination of zinc and lipoic acid. In particular, the cosmetic uses are intended for the treatment of skin stressed by aging (antiaging agent) or by external factors (antipollutant agent).

The hydrogels can also be used in aesthetic medicine applications or mesotherapy.

For the applications and uses specified herein, the hydrogels can be used in combination with substances such as lidocaine, vitamins and amino acids.

The examples below illustrate the invention in greater detail.

### EXAMPLES

### Methods

### Instrumentation Used:

VARIAN VNMR 500 MHz spectrometer equipped with a 5 mm multinuclear reverse probe with a z gradient for determination of the degree of substitution (DS);
Anton Paar MCR 301 rheometer equipped with parallel plates (diameter 25 mm, satin-finish) thermostated to 25° C.

### Degree of Substitution (DS)

The degree of substitution in lipoic esters on the hyaluronic acid derivative was quantitated by NMR spectroscopy. The ¹H NMR spectra were effected in D₂O with a VARIAN VNMR 500 MHz spectrometer equipped with a 5 mm multinuclear reverse probe with a z gradient. The tests were conducted by thermostating the measurement probe to 298° K.

The quantitation of DS in lipoic ester was performed after exhaustive hydrolysis with NaOD directly in the NMR tube.

The ¹H NMR spectrum of the hydrolysate allows integration of the signals attributable to lipoic acid (methylene and methine protons) and those attributable to hyaluronic acid (two anomeric protons); their ratio determines the degree of substitution.

### Determination of Elastic and Viscous Moduli by Rheology Testing.

The rheology testing of the gels was conducted with an Anton Paar MCR 301 rheometer equipped with parallel plates (diameter 25 mm, satin-finish) thermostated to 25° C.

The mechanical spectrum was recorded for each gel in oscillation mode (stress sweep) at a constant frequency of 1 Hz, which allowed the determination of modulus of elasticity G′ and modulus of viscosity G″ (unit of measurement Pa); for some gels the flow curve, which measures viscosity η (unit of measurement Pa*s) on variation of the force applied, was also recorded.

### Example 1: Synthesis of Sodium Hyaluronate Lipoate/Formate (MW: 1500 kDa; DSₗᵢₚ: 0.4; DS_{for}: 0.02)

100 ml of formamide and 5 g of HANa with a molecular weight of 1500 kDa are introduced into a 1-litre reactor. The mixture is thermostated at 95° C. and maintained under stirring at a constant temperature for 1 h, until the polymer is completely dissolved. The temperature is then reduced to 25° C., and the mixture is maintained under stirring overnight.

The next day the temperature is increased to 40° C.; sodium carbonate (Na₂CO₃-264 mg) is then added, and after 0.5 h a solution of lipoyl-imidazolide in acetone (24.0 g; 20%) is added in about 1.5 h. The mixture is left under stirring for 0.5 h at the same temperature. The thermostat is switched off, the reaction is quenched by adding 40 ml acid water, and the product is isolated by precipitation with acetone and subsequent vacuum filtration.

The crude reaction product is purified by several washes with acetone and methanol, each followed by vacuum filtration. The precipitate is dried under vacuum at room temperature for about 17 h.

10 mg of sample is solubilised in 0.9 ml of deuterium oxide (D₂O) and transferred to an NMR test tube.

After hydrolysis of the lipoic and formic esters by adding NaOD (deuterated sodium hydroxide), the NMR spectra exhibit a DS of 0.4 in lipoic acid and 0.02 in formic acid.

### Example 2: Synthesis of Sodium Hyaluronate Lipoate/Formate (MW: 300 kDa; DSₗᵢₚ: 0.5; DS_{for}: 0.03)

100 ml of formamide and 10.05 g of HANa with a molecular weight of 300 kDa are introduced into a 1-litre reactor. The mixture is thermostated at 95° C. and maintained under stirring at a constant temperature for 1 h, until the polymer is completely dissolved. The temperature is then reduced to 25° C., and the mixture is maintained under stirring overnight.

The next day the temperature is increased to 40° C.; sodium carbonate (Na₂CO₃-528 mg) is then added, and after 0.5 h a solution of lipoyl-imidazolide in acetone (25.0 g; 20%) is added in about 1.5 h. The mixture is left under stirring for 0.5 h at the same temperature. The reaction is quenched by adding 80 ml acid water, and the product is isolated by precipitation with acetone and subsequent vacuum filtration.

The crude reaction product is purified by several washes with acetone and methanol, each followed by vacuum filtration. The precipitate is dried under vacuum at room temperature for about 6 h.

10 mg of sample is solubilised in 0.9 ml of deuterium oxide (D₂O) and transferred to an NMR test tube.

After hydrolysis of the lipoic and formic esters by adding NaOD (deuterated sodium hydroxide), the NMR spectra exhibit a DS of 0.5 in lipoic acid and 0.03 in formic acid.

### Example 3: Synthesis of Sodium Hyaluronate Lipoate/Formate (MW: 50 kDa; DSₗᵢₚ: 0.5; DS_{for}: 0.03)

100 ml of formamide and 10.0 g of HANa with a molecular weight of 50 kDa are introduced into a 1-litre reactor. The mixture is thermostated at 95° C. and maintained under stirring at a constant temperature for 1 h, until the polymer is completely dissolved. The temperature is then reduced to 25° C., and the mixture is maintained under stirring overnight.

The next day the temperature is increased to 40° C.; sodium carbonate (Na₂CO₃-500 mg) is then added, and after 0.5 h a solution of lipoyl-imidazolide in DMSO (20.0 g; 20%) is added in about 1.5 h. The mixture is left under stirring for 0.5 h at the same temperature. The reaction is quenched by adding 40 ml acid water, and the product is isolated by precipitation with acetone and subsequent vacuum filtration.

The crude reaction product is purified by several washes with acetone and methanol, each followed by vacuum filtration. The precipitate is dried under vacuum at room temperature for about 18 h.

10 mg of sample is solubilised in 0.9 ml of deuterium oxide (D₂O) and transferred to an NMR test tube.

After hydrolysis of the lipoic and formic esters by adding NaOD (deuterated sodium hydroxide), the NMR spectra exhibit a DS of 0.5 in lipoic acid and 0.03 in formic acid.

### Example 4: Synthesis of Sodium Hyaluronate Lipoate/Formate (MW: 1500 kDa; DSₗᵢₚ: 0.3; DS_{for}: 0.02)

200 ml of formamide and 10.0 g of HANa with a molecular weight of 1500 kDa are introduced into a 1-litre reactor. The mixture is thermostated at 95° C. and maintained under stirring at a constant temperature for 1 h, until the polymer is completely dissolved. The temperature is then reduced to 25° C., and the mixture is maintained under stirring overnight.

The next day the temperature is increased to 40° C.; sodium carbonate (Na₂CO₃-527 mg) is then added, and after 0.5 h a solution of lipoyl-imidazolide in acetone (47.2 g; 20%) is added in about 1.75 h. The mixture is left under stirring for 0.5 h at the same temperature. The reaction is quenched by adding 80 ml acid water, and the product is isolated by precipitation with acetone and subsequent vacuum filtration.

The crude reaction product is purified by several washes with acetone and methanol, each followed by vacuum filtration. The precipitate is dried under vacuum at room temperature for about 18 h.

10 mg of sample is solubilised in 0.9 ml of deuterium oxide (D₂O) and transferred to an NMR test tube.

After hydrolysis of the lipoic and formic esters by adding NaOD (deuterated sodium hydroxide), the NMR spectra exhibit a DS of 0.3 in lipoic acid and 0.02 in formic acid.

### Example 5: Synthesis of Sodium Hyaluronate Lipoate/Formate (MW: 300 kDa; DSₗᵢₚ: 0.3; DS_{for}: 0.01)

160 ml of formamide and 8 g of HANa with a molecular weight of 300 kDa are introduced into a 1-litre reactor. The mixture is thermostated at 95° C. and maintained under stirring at a constant temperature for 1.5 h, until the polymer is completely dissolved. The temperature is then reduced to 25° C., and the mixture is maintained under stirring overnight.

The next day sodium carbonate (Na₂CO₃-400 mg) is added, and after 0.5 h a solution of lipoyl-imidazolide in acetone (15 g; 20%) is added in about 1.75 h. The mixture is left under stirring for 1 h at the same temperature. The reaction is quenched by adding 16 ml acid water, and the product is isolated by precipitation with acetone and subsequent vacuum filtration.

The crude reaction product is purified by several washes with acetone and methanol, each followed by vacuum filtration. The precipitate is dried under vacuum at room temperature for about 18 h.

10 mg of sample is solubilised in 0.9 ml of deuterium oxide (D₂O) and transferred to an NMR test tube.

After hydrolysis of the lipoic and formic esters by adding NaOD (deuterated sodium hydroxide), the NMR spectra exhibit a DS of 0.3 in lipoic acid and 0.01 in formic acid.

### Example 6: Synthesis of Sodium Hyaluronate Lipoate/Formate (MW: 50 kDa; DSₗᵢₚ: 0.3; DS_{for}: 0.01)

30 ml of formamide and 3 g of HANa with a molecular weight of 50 kDa are introduced into a 500 ml 3-necked flask. The mixture is thermostated at 95° C. and maintained under stirring at a constant temperature for 1 h, until the polymer is completely dissolved. The temperature is then reduced to 25° C., and the mixture is maintained under stirring overnight.

The next day the temperature is increased to 40° C.; sodium carbonate (Na₂CO₃-150 mg) is then added, and after 0.5 h a solution of lipoyl-imidazolide in DMSO (15.0 g; 20%) is added in about 1.5 h. The mixture is left under stirring for 0.5 h at the same temperature. The reaction is quenched by adding 10 ml acid water, and the product is isolated by precipitation with acetone and subsequent vacuum filtration.

The crude reaction product is purified by several washes with acetone and methanol, each followed by vacuum filtration. The precipitate is dried under vacuum at room temperature for about 18 h.

10 mg of sample is solubilised in 0.9 ml of deuterium oxide (D₂O) and transferred to an NMR test tube.

After hydrolysis of the lipoic and formic esters by adding NaOD (deuterated sodium hydroxide), the NMR spectra exhibit a DS of 0.3 in lipoic acid and 0.01 in formic acid.

### Example 7: Synthesis of Sodium Hyaluronate Lipoate/Formate (80 MW 1500 kDa: 20 MW 300 kDa; DSₗᵢₚ: 0.3; DS_{for}: 0.02)

100 ml of formamide followed by 4 g of HANa with a molecular weight of 1500 kDa and 1 g of HANa with a molecular weight of 300 kDa are introduced into a 1 litre reactor. The mixture is thermostated at 90° C. and maintained under stirring at a constant temperature for 1 h, until the polymer is completely dissolved. The temperature is then reduced to 25° C., and the mixture is maintained under stirring overnight.

The next day the temperature is increased to 40° C.; sodium carbonate (Na₂CO₃-264 mg) is then added, and after 0.5 h a solution of lipoyl-imidazolide in acetone (31.5 g; 20%) is added in about 1.5 h. The mixture is left under stirring for 0.5 h at the same temperature. The reaction is quenched by adding 40 ml acid water, and the product is isolated by precipitation with acetone and subsequent vacuum filtration.

The crude reaction product is purified by several washes with acetone and methanol, each followed by vacuum filtration. The precipitate is dried under vacuum at room temperature for about 19 h.

10 mg of sample is solubilised in 0.9 ml of deuterium oxide (D₂O) and transferred to an NMR test tube.

After hydrolysis of the lipoic and formic esters by adding NaOD (deuterated sodium hydroxide), the NMR spectra exhibit a DS of 0.3 in lipoic acid and 0.02 in formic acid.

### Example 8: Synthesis of Sodium Hyaluronate Lipoate/Formate (20 MW 1500 kDa: 80 MW 300 kDa; DSₗᵢₚ: 0.3; DS_{for}: 0.02)

100 ml of formamide followed by 1 g of HANa with a molecular weight of 1500 kDa and 4 g of HANa with a molecular weight of 300 kDa are introduced into a 1 litre reactor. The mixture is thermostated at 95° C. and maintained under stirring at a constant temperature for 1 h, until the polymer is completely dissolved. The temperature is then reduced to 25° C., and the mixture is maintained under stirring overnight.

The next day the temperature is increased to 40° C.; sodium carbonate (Na₂CO₃-264 mg) is then added, and after 0.5 h a solution of lipoyl-imidazolide in acetone (31.5 g; 20%) is added in about 2.5 h. The reaction is quenched by adding 40 ml acid water, and the product is isolated by precipitation with acetone and subsequent vacuum filtration.

The crude reaction product is purified by several washes with acetone and methanol, each followed by vacuum filtration. The precipitate is dried under vacuum at room temperature for about 19 h.

10 mg of sample is solubilised in 0.9 ml of deuterium oxide (D₂O) and transferred to an NMR test tube.

After hydrolysis of the lipoic and formic esters by adding NaOD (deuterated sodium hydroxide), the NMR spectra exhibit a DS of 0.3 in lipoic acid and 0.02 in formic acid.

### Example 9: Preparation of Hydrogel Containing Sodium Hyaluronate Lipoate/Formate Zinc Gluconate Complex (2 w/V; MW: 1500 kDa; DSₗᵢₚ: 0.4; DS_{for}: 0.02; Zn 0.1 mM)

100 ml of injectable water, 4.6 mg of zinc gluconate and 0.9 g of NaCl are introduced into a 200 ml flask, and 2 g of the sample of Example 1 is added to the mixture. The system is mixed with an Ultra-turrax.

After centrifugation the mixture is dispensed into syringes. A gel with the following characteristics is obtained: ηₘₐₓ=68000 Pa*s; G′: 399.6 Pa; G″: 88.7 Pa.

Some syringes undergo a sterilising cycle in the autoclave (121° C.; 15 min). A gel with the following characteristics is obtained: ηₘₐₓ=42000 Pa*s; G′: 98.1 Pa; G″: 29.7 Pa.

### Example 10: Preparation of Hydrogel Containing Sodium Hyaluronate Lipoate/Formate Zinc Gluconate Complex (2 w/V; MW: 300 kDa; DSₗᵢₚ: 0.5; DS_{for}: 0.03; Zn 0.1 mM)

100 ml of injectable water, 4.5 mg of zinc gluconate and 0.9 g of NaCl are introduced into a 200 ml flask, and 2 g of the sample of Example 2 is added to the mixture. The system is mixed with an Ultra-turrax.

After centrifugation the mixture is dispensed into syringes. A gel with the following characteristics is obtained: G′: 117.9; Pa; G″: 33.1 Pa.

Some syringes undergo a sterilising cycle in the autoclave (121° C.; 15 min). A gel with the following characteristics is obtained: ηₘₐₓ=200000 Pa*s; G′: 134.3 Pa; G″: 7.7 Pa.

### Example 11: Preparation of Hydrogel Containing Sodium Hyaluronate Lipoate/Formate Zinc Gluconate Complex (2 w/V; MW: 50 kDa; DSₗᵢₚ: 0.5; DS_{for}: 0.03; Zn 0.1 mM)

100 ml of injectable water, 4.5 mg of zinc gluconate and 0.9 g of NaCl are introduced into a 200 ml flask, and 2 g of the sample of Example 3 is added to the mixture. The system is mixed with an Ultra-turrax.

After centrifugation the mixture is dispensed into syringes.

### Example 12: Preparation of Hydrogel Containing Sodium Hyaluronate Lipoate/Formate Zinc Gluconate Complex (2 w/V; MW: 1500 kDa; DSₗᵢₚ: 0.4; DS_{for}: 0.02; Zn 0.2 mM)

100 ml of injectable water, 9.1 mg of zinc gluconate and 0.9 g of NaCl are introduced into a 200 ml flask, and 2 g of the sample of Example 1 is added to the mixture. The system is mixed with an Ultra-turrax.

After centrifugation the mixture is dispensed into syringes.

Some syringes undergo a sterilising cycle in the autoclave (121° C.; 15 min.). A gel with the following characteristics is obtained: ηₘₐₓ=40000 Pa*s; G′: 46.8 Pa; G″: 12.6 Pa.

### Example 13: Preparation of Hydrogel Containing Sodium Hyaluronate Lipoate/Formate Zinc Gluconate Complex (2 w/V; MW: 300 kDa; DSₗᵢₚ: 0.5; DS_{for}: 0.03; Zn 0.2 mM)

100 ml of injectable water, 9.1 mg of zinc gluconate and 0.9 g of NaCl are introduced into a 200 ml flask, and 2 g of the sample of Example 2 is added to the mixture. The system is mixed with an Ultra-turrax.

After centrifugation the mixture is dispensed into syringes.

### Example 14: Preparation of Hydrogel Containing Sodium Hyaluronate Lipoate/Formate Zinc Gluconate Complex (MW: 50 kDa; DSₗᵢₚ: 0.5; DS_{for}: 0.03; Zn 0.2 mM)

100 ml of injectable water, 9.2 mg of zinc gluconate and 0.9 g of NaCl are introduced into a 200 ml flask, and 2 g of the sample of Example 3 is added to the mixture. The system is mixed with an Ultra-turrax.

After centrifugation the mixture is dispensed into syringes.

### Example 15: Preparation of Hydrogel Containing Sodium Hyaluronate Lipoate/Formate Zinc Gluconate Complex (2 w/V; MW: 1500 kDa; DSₗᵢₚ: 0.4; DS_{for}: 0.02; Zn 0.15 mM)

100 ml of injectable water, 6.8 mg of zinc gluconate and 0.9 g of NaCl are introduced into a 200 ml flask, and 2 g of the sample of Example 1 is added to the mixture. The system is mixed with an Ultra-turrax.

After centrifugation the mixture is dispensed into syringes.

Some syringes undergo a sterilising cycle in the autoclave (121° C.; 15 min). A gel with the following characteristics is obtained: ηₘₐₓ=16500 Pa*s; G′: 17.9 Pa; G″: 6.3 Pa.

### Example 16: Preparation of Hydrogel Containing Sodium Hyaluronate Lipoate/Formate Zinc Gluconate Complex (2 w/V; 50:50; MW: 1500 kDa; DSₗᵢₚ: 0.3; DS_{for}: 0.02; +MW: 300 kDa; DSₗᵢₚ: 0.5; DS_{for}: 0.03; Zn 0.1 mM)

100 ml of injectable water, 4.6 mg of zinc gluconate, 0.9 g of NaCl and 0.3 g of lidocaine are introduced into a 200 ml flask. 1 g of the sample of Example 4 and 1 g of the sample of Example 2 are added to the mixture. The system is mixed with an Ultra-turrax.

After centrifugation the mixture is dispensed into syringes, which undergo a sterilising cycle in the autoclave (121° C.; 15 min). A gel with the following characteristics is obtained: ηₘₐₓ=2400 Pa*s; G′: 19.6 Pa; G″: 5.5 Pa.

### Example 17: Preparation of Hydrogel Containing Sodium Hyaluronate Lipoate/Formate Zinc Gluconate Complex (2 w/V; 20:80; MW: 1500 kDa; DSₗᵢₚ: 0.3; DS_{for}: 0.02; +MW: 300 kDa; DSₗᵢₚ: 0.5; DS_{for}: 0.03; Zn 0.1 mM)

100 ml of injectable water, 4.6 mg of zinc gluconate, 0.9 g of NaCl and 0.3 g of lidocaine are introduced into a 200 ml flask. 0.4 g of the sample of Example 4 and 1.6 g of the sample of Example 2 are added to the mixture. The system is mixed with an Ultra-turrax.

After centrifugation the mixture is dispensed into syringes, which undergo a sterilising cycle in the autoclave (121° C.; 15 min). A gel with the following characteristics is obtained: ηₘₐₓ=57,500 Pa*s; G′: 53.4 Pa; G″: 5.7 Pa.

### Example 18: Preparation of Hydrogel Containing Sodium Hyaluronate Lipoate/Formate Zinc Gluconate Complex (2 w/V; 50:50; MW: 1500 kDa; DSₗᵢₚ: 0.3; DS_{for}: 0.02; +MW: 300 kDa; DSₗᵢₚ: 0.5; DS_{for}: 0.03; Zn 0.2 mM)

100 ml of injectable water, 9.2 mg of zinc gluconate and 0.9 g of NaCl are introduced into a 200 ml flask. 1 g of the sample of Example 4 and 1 g of the sample of Example 2 are added to the mixture. The system is mixed with an Ultra-turrax.

After centrifugation the mixture is dispensed into syringes, which undergo a sterilising cycle in the autoclave.

### Example 19: Preparation of Hydrogel Containing Sodium Hyaluronate Lipoate/Formate Zinc Gluconate Complex (2 w/V; DSₗᵢₚ: 0.3; DS_{for}: 0.02; Zn 0.2 mM)

100 ml of injectable water, 9.1 mg of zinc gluconate, 0.9 g of NaCl and 0.3 g of lidocaine are introduced into a 200 ml flask. 2 g of the sample of Example 8 is added to the mixture. The system is mixed with an Ultra-turrax.

After centrifugation the mixture is dispensed into syringes, which undergo a sterilising cycle in the autoclave (121° C.; 15 min). A gel with the following characteristics is obtained: ηₘₐₓ: 1800 PA*s; G′: 6.0 Pa; G″: 5.2 Pa.

### Example 20: Preparation of Hydrogel Containing Sodium Hyaluronate Lipoate/Formate Zinc Gluconate Complex (80:20 MW: 1500 kDa; DSₗᵢₚ: 0.3; DS_{for}: 0.02; +MW: 300 kDa; DSₗᵢₚ: 0.5; DS_{for}: 0.03; Zn 0.1 mM)

100 ml of injectable water, 4.5 mg of zinc gluconate, 0.9 g of NaCl and 0.3 g of lidocaine are introduced into a 200 ml flask. 1.6 g of the sample of Example 4 and 0.4 g of the sample of Example 2 are added to the mixture. The system is mixed with an Ultra-turrax.

After centrifugation the mixture is dispensed into syringes.

### Example 21: Preparation of Hydrogel Containing Sodium Hyaluronate Lipoate/Formate Zinc Gluconate Complex (2 w/V; DSₗᵢₚ: 0.3; DS_{for}: 0.02; Zn 0.2 mM)

100 ml of injectable water, 9.2 mg of zinc gluconate, 0.9 g of NaCl and 0.3 g of lidocaine are introduced into a 200 ml flask. 2 g of the sample of Example 7 is added to the mixture. The system is mixed with an Ultra-turrax.

After centrifugation the mixture is dispensed into syringes

### Example 22: Preparation of Hydrogel Containing Sodium Hyaluronate Lipoate/Formate Zinc Gluconate Complex (4 w/V; DSₗᵢₚ: 0.3; DS_{for}: 0.02; Zn 0.2 mM)

100 ml of injectable water, 9.2 mg of zinc gluconate, 0.9 g of NaCl and 0.3 g of lidocaine are introduced into a 200 ml flask. 4 g of the sample of Example 7 is added to the mixture. The system is mixed with an Ultra-turrax.

After centrifugation the mixture is dispensed into syringes, which undergo a sterilising cycle in the autoclave (121° C.; 15 min). A gel with the following characteristics is obtained: ηₘₐₓ=58800 Pa*s; G′: 118.1 Pa; G″: 40.0 Pa.

### Example 23: Preparation of Hydrogel Containing Sodium Hyaluronate Lipoate/Formate Zinc Gluconate Complex (6 w/V; DSₗᵢₚ: 0.3; DS_{for}: 0.02; Zn 0.2 mM)

100 ml of injectable water, 9.2 mg of zinc gluconate, 0.9 g of NaCl and 0.3 g of lidocaine are introduced into a 200 ml flask. 6 g of the sample of Example 7 is added to the mixture. The system is mixed with an Ultra-turrax.

After centrifugation the mixture is dispensed into syringes, which undergo a sterilising cycle in the autoclave (121° C.; 15 min). A gel with the following characteristics is obtained: ηₘₐₓ=60000 Pa*s; G′: 184.2 Pa; G″: 81.2 Pa.

### Example 24: Preparation of Hydrogel Containing Sodium Hyaluronate Lipoate/Formate Zinc Gluconate Complex (4 w/V; DSₗᵢₚ: 0.3; DS_{for}: 0.02; Zn 0.2 mM)

100 ml of injectable water, 9.2 mg of zinc gluconate, 0.9 g of NaCl and 0.3 g of lidocaine are introduced into a 200 ml flask. 4 g of the sample of Example 8 is added to the mixture. The system is mixed with an Ultra-turrax.

After centrifugation the mixture is dispensed into syringes, which undergo a sterilising cycle in the autoclave (121° C.; 15 min). A gel with the following characteristics is obtained: ηₘₐₓ=57000 Pa*s; G′: 122.9 Pa; G″: 35.4 Pa.

### Example 25: Preparation of Hydrogel Containing Sodium Hyaluronate Lipoate/Formate Zinc Gluconate Complex (6 w/V; DSₗᵢₚ: 0.3; DS_{for}: 0.02; Zn 0.2 mM)

100 ml of injectable water, 9.2 mg of zinc gluconate, 0.9 g of NaCl and 0.3 g of lidocaine are introduced into a 200 ml flask. 6 g of the sample of Example 8 is added to the mixture. The system is mixed with an Ultra-turrax.

After centrifugation the mixture is dispensed into syringes, which undergo a sterilising cycle in the autoclave (121° C.; 15 min). A gel with the following characteristics is obtained: ηₘₐₓ=170000 Pa*s; G′: 283.0 Pa; G″: 84.3 Pa.

### Example 26: Preparation of Hydrogel Containing Sodium Hyaluronate Lipoate/Formate Zinc Gluconate Complex (2 w/V; 80:20 MW: 1500 kDa; DSₗᵢₚ: 0.3; DS_{for}: 0.02; +MW: 300 kDa; DSₗᵢₚ: 0.5; DS_{for}: 0.03; Zn 0.5 mM)

100 ml of injectable water, 22.5 mg of zinc gluconate, 0.9 g of NaCl and 0.3 g of lidocaine are introduced into a 200 ml flask. 1.6 g of the sample of Example 4 and 0.4 g of the sample of Example 2 are added to the mixture. The system is mixed with an Ultra-turrax.

After centrifugation the mixture is dispensed into syringes.

### Example 27: Preparation of Hydrogel Containing Sodium Hyaluronate Lipoate/Formate Zinc Gluconate Complex (2 w/V; MW: 300 kDa; DSₗᵢₚ: 0.5; DS_{for}: 0.03; Zn 10 mM)

100 ml of injectable water, 455 mg of zinc gluconate and 0.9 g of NaCl are introduced into a 200 ml flask. 2 g of the sample of Example 2 is added to the mixture. The system is mixed with an Ultra-turrax.

After centrifugation the mixture is dispensed into syringes. A gel with the following characteristics is obtained: ηₘₐₓ: 200000 Pa*s; G′: 314 Pa; G″: 29 Pa.

Some syringes undergo a sterilising cycle in the autoclave (121° C.; 15 min). A gel with the following characteristics is obtained: ηₘₐₓ=130000 Pa*s; G′: 106.5 Pa; G″: 9.9 Pa.

### Example 28: Preparation of Hydrogel Containing Sodium Hyaluronate Lipoate/Formate Zinc Gluconate Complex (2 w/V; MW: 1500 kDa; DSₗᵢₚ: 0.3; DS_{for}: 0.02; Zn 0.5 mM)

100 ml of injectable water, 22.5 mg of zinc gluconate and 0.9 g of NaCl are introduced into a 200 ml flask. 2 g of the sample of Example 4 is added to the mixture. The system is mixed with an Ultra-turrax.

After centrifugation the mixture is dispensed into syringes.

### Example 29: Preparation of Hydrogel Containing Sodium Hyaluronate Lipoate/Formate Zinc Gluconate Complex (2 w/V; MW: 1500 kDa; DSₗᵢₚ: 0.4; DS_{for}: 0.02; Zn 0.5 mM)

100 ml of injectable water, 22.5 mg of zinc gluconate and 0.9 g of NaCl are introduced into a 200 ml flask. 2 g of the sample of Example 1 is added to the mixture. The system is mixed with an Ultra-turrax.

After centrifugation the mixture is dispensed into syringes

### Example 30: Preparation of Hydrogel Containing Sodium Hyaluronate Lipoate/Formate Zinc Gluconate Complex (2 w/V; MW: 50 kDa; DSₗᵢₚ: 0.3; DS_{for}: 0.01; Zn 0.1 mM)

100 ml of injectable water, 4.5 mg of zinc gluconate and 0.9 g of NaCl are introduced into a 200 ml flask. 2 g of the sample of Example 6 is added to the mixture. The system is mixed with an Ultra-turrax.

After centrifugation the mixture is dispensed into syringes.

### Example 31: Preparation of Hydrogel Containing Sodium Hyaluronate Lipoate/Formate Zinc Gluconate Complex (2 w/V; MW: 1500 kDa; DSₗᵢₚ: 0.3; DS_{for}: 0.02; Zn 0.1 mM)

100 ml of injectable water, 4.5 mg of zinc gluconate and 0.9 g of NaCl are introduced into a 200 ml flask. 2 g of the sample of Example 4 is added to the mixture. The system is mixed with an Ultra-turrax.

After centrifugation the mixture is dispensed into syringes.

### Example 32: Synthesis of Sodium Hyaluronate Lipoate (MW: 1500 kDa; DSₗᵢₚ: 0.45; DS_{for}: 0.0)

200 ml of formamide and 5 g of HANa with a molecular weight of 1500 kDa are introduced into a 1-litre reactor. The mixture is thermostated at 75° C. and maintained under stirring at a constant temperature for 1 h, until the polymer is completely dissolved. The temperature is then reduced to 25° C., and the mixture is maintained under stirring overnight.

The next day the temperature is increased to 40° C.; sodium carbonate (Na₂CO₃-660 mg) is then added, and after 0.5 h a solution of lipoyl-imidazolide in acetone (46.9 g; 20%) is added in about 1.5 h. The mixture is left under stirring for 0.5 h at the same temperature. The reaction is quenched by adding 40 ml of acidic water, and the product is isolated by precipitation with acetone and subsequent vacuum filtration.

The crude reaction product is purified by several washes with acetone and methanol, each followed by vacuum filtration. The precipitate is dried under vacuum at room temperature for about 19 h.

10 mg of sample is solubilised in 0.9 ml of deuterium oxide (D₂O) and transferred to an NMR test tube.

After hydrolysis of the lipoic and formic esters by adding NaOD (deuterated sodium hydroxide), the NMR spectra show a DS of 0.45 in lipoic acid and a DS of 0.0 in formic acid.

### Example 33: Preparation of Hydrogel Containing Sodium Hyaluronate Lipoate/Formate Zinc Gluconate Complex (2 w/V; DSₗᵢₚ: 0.3; DS_{for}: 0.0; Zn 0.2 mM)

100 ml of injectable water, 9.2 mg of zinc gluconate, 0.9 g of NaCl and 0.3 g of lidocaine are introduced into a 200 ml flask. 2 g of the sample of Example 32 is added to the mixture. The system is mixed with an Ultra-turrax.

After centrifugation the mixture is dispensed into syringes.

### Example 34: Enzymatic Degradation of a Hydrogel Containing Sodium Hyaluronate Lipoate/Formate Zinc Gluconate Complex (2 w/V; MW: 1500 kDa; DSₗᵢₚ: 0.4; DS_{for}: 0.02; Zn 0.1 mM) and of a Commercial Hydrogel Based on Sodium Hyaluronate Crosslinked With BDDE

The resistance to enzymatic degradation of a hydrogel prepared as indicated in example 9 (syringe containing 2 ml of gel 2 w/v; MW: 1500 kDa; DSₗᵢₚ: 0.4; DS_{for}: 0.02; Zn 0.1 mM, sterilised by heat cycle of 121° C. for 15 min) was evaluated according to the degradation kinetics by measuring the reduction in the modulus of elasticity (G′) over time. 0.5 ml of gel was extruded from the syringe directly onto the lower plate of the rheometer, and modulus of elasticity G′ was evaluated at a constant frequency (1 Hz) and force (1 Pa) at the temperature of 25° C. The breakdown kinetics were conducted by adding 50 μl of a solution of bovine testicular hyaluronidase in 30 mM acetate buffer pH 5.5 (activity 1500 U/ml) to 0.5 ml of gel. The reduction in modulus of elasticity G′ over a time indicative of the cutting of the polymer chains was evaluated, and the breakdown kinetics were conducted under the same experimental conditions for comparison purposes, using a common hydrogel crosslinked with BDDE (1,4-butanediol diglycidyl ether) available on the market in 1 ml syringes at the concentration of 2% in phosphate-buffered saline pH 7. The modulus of elasticity 60 minutes after addition of the hyaluronidase, indicated as G′₆₀, was used as comparison parameter. Table 1 below shows the measured values of G′, specifying the resistance to enzymatic degradation and residual modulus of elasticity after 60 min compared with the initial modulus.

TABLE 1

| Hydrogel 2 | G′_{zero} [Pa] | G′₆₀ [Pa] | Residual G′ |
|---|---|---|---|
| Example 9 | 95 | 38 | 41 |
| Commercial product crosslinked | 79 | 7 | 9 |
| with BDDE | | | |

The following are embodiments of the invention.

Embodiment 1: A hydrogel containing:
hyaluronic acid esterified on its free hydroxyls with lipoic acid or with lipoic acid and formic acid, or a pharmaceutically acceptable salt thereof, and
zinc gluconate.
   Embodiment 2: A hydrogel according to embodiment 1, wherein the hyaluronic acid has a molecular weight ranging from 1 kDa to 4×10³ kDa.
   Embodiment 3: A hydrogel according to embodiments 1-2, wherein the number of lipoic acid residues per GlcNAc-GlcUA disaccharide unit of hyaluronic acid ranges from 0.01 to 0.5.
   Embodiment 4: A hydrogel according to embodiments 1-3, wherein the number of formic acid residues per GlcNAc-GlcUA disaccharide unit of hyaluronic acid ranges from 0 to 0.1.
   Embodiment 5: A hydrogel according to embodiments 1-4, wherein the esterified hyaluronic acid salt is the sodium salt.
   Embodiment 6: A hydrogel according to embodiments 1-5, wherein the amount of zinc gluconate ranges from 0.1% to 25% by weight of the sodium hyaluronate lipoate or sodium hyaluronate lipoate and formate.
   Embodiment 7: A pharmaceutical or cosmetic composition or medical device containing a hydrogel according to embodiments 1-6, optionally in combination with biologically active compounds.
   Embodiment 8: A composition or device according to embodiment 7, wherein said compounds are selected from lidocaine, vitamins and amino acids.
   Embodiment 9: A composition according to embodiments 7-8, which is in a form suitable for topical administration selected from ocular application; injectable administration, particularly intradermal, mesodermal, intra-articular or intraocular injection; and ophthalmic administration.
   Embodiment 10: A hydrogel according to embodiments 1-6 or a composition according to embodiments 7-9, for use in the treatment of dermatological disorders, particularly acne, erythema and burns, or in the treatment of joint inflammation.
   Embodiment 11: The use of a hydrogel according to claims 1-6 or of the composition according to claims 7-9, to treat skin blemishes or as antiaging or antipollutant agent.

1. A process for the preparation of a hydrogel containing hyaluronic acid esterified on its free hydroxyls with lipoic acid or with lipoic acid and formic acid, or a pharmaceutically acceptable salt thereof, and zinc gluconate, which comprises the following steps:
(a) mixing in water of zinc gluconate and esterified hyaluronic acid or a salt thereof, until a viscous solution is obtained; and
(b) leaving the viscous solution obtained in (a) to stand for a time ranging from 1 to 48 hours and obtaining said hydrogel.

2. The process according to claim 1, further comprising sterilizing the hydrogel.

3. The process according to claim 2, wherein the sterilizing step takes place in the autoclave.

4. The process according to claim 3, wherein the sterilizing step takes place at 121° C. for 15 min.

5. The process according to claim 1, wherein said mixing in water is carried out at a temperature ranging from 20° C. to 30° C.

6. The process according to claim 1, wherein the hyaluronic acid has a molecular weight ranging from 1kDa to 4×10³ kDa.

7. The process according to claim 1, wherein the number of lipoic acid residues per GlcNAc-GlcUA disaccharide unit of hyaluronic acid ranges from 0.01 to 0.5

8. The process according to claim 1, wherein the number of formic acid residues per GlcNAc-GlcUA disaccharide unit of hyaluronic acid ranges from 0 to 0.1.

9. The process according to claim 1, wherein the esterified hyaluronic acid salt is sodium salt.

10. The process according to claim 1, wherein the amount of zinc gluconate ranges from 0.1% to 25% by weight of sodium hyaluronate lipoate or sodium hyaluronate lipoate and formate.
